(19) United States
(12) Patent Application Publication
(10) Pub. No.: US 2025/0241681 A1
(43) Pub. Date: Jul. 31, 2025

(54) INTAUTERINE DELIVERY DEVICE, SYSTEM AND METHOD THEREOF

(71) Applicant: INTIMO MEDICAL LTD, Nazareth (IL)

(72) Inventor: Matan SHABAT, Eilat (IL)

(21) Appl. No.: 18/855,732

(22) PCT Filed: Apr. 16, 2023
(86) PCT No.: PCT/IL2023/050398

§ 371 Date: Oct. 10, 2024

(30) Foreign Application Priority Data
Apr. 14, 2022 (IL) 292298

(51) Int. Cl. A61B 17/43; A61B 17/00; A61B 34/20; A61B 34/30

(52) U.S. Cl. CPC A61B 17/43; A61B 34/20; A61B 2017/00057; A61B 2034/302

(57) ABSTRACT

An intrauterine insemination (IUI) device having a sampling channel, a sample fluid flow module, an electronics module, and a sensor module the device configured to perform an analysis of a sample that is associated with the sampling channel.

### FIELD OF THE INVENTION

The present invention relates to a device and system for intrauterine delivery of fluids and in particular, to such a device and system providing improved fluid delivery to the uterus for artificial insemination.

### BACKGROUND OF THE INVENTION

Artificial insemination attempts to assist fertility by introducing semen directly into the uterine cavity, generally near the opening of the fallopian tube. This is accomplished with a dedicated catheter known as an IntraUterine Insemination (‘IUI’) catheter. An IUI catheter introduced semen past the cervix so as to increase likelihood of in utero fertilization.

State of the art IUI catheters are simple catheters that function as a transport tube.

### SUMMARY OF THE INVENTION

The present invention overcomes the deficiencies of the background art by providing a system and device for improving the likelihood and/or improving the success rate of current IUI catheters.

Embodiments of the present invention provide an intrauterine insemination IUI device and associated system. The IUI device comprise a body, a sampling channel, a fluid flow module, an electronics module, and a sensor module. In embodiments the IUI device may form a system comprising the IUI device according to embodiments that is in communication with a data processing center.

Embodiments of the present invention provide a method for improving with the IUI device according to the present embodiments the method comprising monitoring and measuring semen parameters, for example including but not limited to semen sample motility, semen sample activity rate, sample viscosity, sample volume, concentration of semen within sample, sample pH, semen morphology, the like or any combination thereof, and adjusting the sample delivery parameters in accordance with the measured sample parameters.

In embodiments, the sample delivery parameters may for example include but is not limited to at least one or more of: delivery rate, delivery speed, delivery location, the like, or any combination thereof.

Embodiments of the present invention for an IUI device comprising a body that is optionally configured as a handle fit with electronics module, a sample channel and a fluid flow module.

In embodiments, electronics module preferably comprises necessary electronic circuitry and processing hardware and/or software to render the IUI device functional.

In embodiments, sample channel may be configured as a channel and/or a catheter for receiving a sample for delivery. In embodiments, the sample channel may be configured to receive a disposable and/or single use catheter and/or sleeve. In embodiments, sample channel may be configured to be a multi-use channel for directly receiving and delivery a semen sample.

In embodiments, a fluid flow module provides for facilitating uptake and delivery of a sample. In embodiments, fluid flow module may comprise a negative pressure sub-module and/or a positive pressure sub-module. In embodiments a negative pressure sub-module may be configured to provide suction and/or vacuum necessary to uptake and/or receive a sample. In embodiments a positive pressure sub-module may be configured to provide positive pressure for the delivery of a sample to its delivery location.

Optionally fluid flow module may for example including but is not limited to at least one or more of: a motors, hydraulic motor, electromagnetic motor, piston, air piston, spring loaded movement, gear, linear spring, windings, electromagnet, pump, compressor, the like or any combination thereof.

In embodiments IUI device may comprise a sensor module comprising at least one or more sensor to render the IUI device functional. In embodiments sensor module may for example include at least one or more sensors for example including but not limited to: image sensor, pH sensor, flow sensor, temperature sensor, pressure sensor, positional sensor, the like or any combination thereof.

In embodiments IUI device according to the present invention may be configured to communicate with a Data Processing Center provided to receive and process data relating to IUI device and treatments thereof. For example, a data processing center may for example be utilized to perform big data analysis wherein such analysis may be utilized to improve success rates, and/or to change treatment protocols, and/or, to undertake artificial intelligence (AI) analysis, and/or, to review all historic data. In some embodiments, data processing center may be utilized to provide advice so as to improve success rate, more preferably substantially in real time.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagrams of an exemplary device and system according to embodiments of the present invention;

FIG. 2 is a schematic illustrative diagram of an exemplary device according to an embodiments of the present invention; and

FIG. 3 is a schematic flow chart depicting a method of use of the device and system according to embodiments of the present invention.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
50 Data Processing Center;
55 Auxiliary devices
100 IUI device;
102 device body;
102d body distal end;
102p body proximal end;
104 fluid flow module;
104a negative pressure sub-module;
104b positive pressure sub-module;
106 sample channel;
110 electronics module;
111 user interface (UI);
112 power module;
114 controller and/or processor module;
116 communication module;
118 memory module;
120 sensor module;
122 temperature sensor;
124 pH sensor;
126 imaging sensor;
128 flow sensor;
150 system;

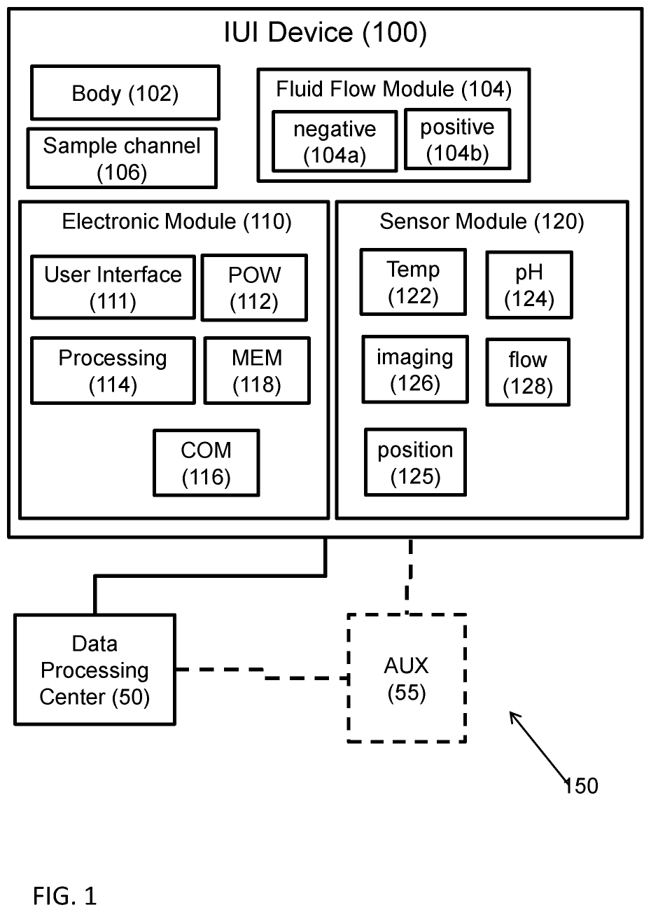

FIG. 1 shows a schematic block diagram of an intrauterine insemination (IUI) device 100 and a system 150 thereof, according to embodiments of the present invention. IUI device 100 preferably utilized to facilitate delivery of a biological fluid, most preferably a semen sample or optionally a sample comprising a fertilized ovum at different stages to an intrauterine location so as to improve likelihood of fertilization and/or implantation.

IUI device 100 comprises a body 102 that is configured to be a housing of at least a portion of device 100. In some embodiments body 102 may be shaped and configured in the form of a handle to be manually operated by a practitioner.

In some embodiments at least a portion of body 102 may be configured and/or shaped to accommodate and/or compliment additional tools and/or devices to facilitate an insemination procedure. For example, a portion of body 102 may be configured to associate with gynecological tools to facilitate placement within the uterus, for example including but not limited to speculum or the like.

In some embodiments body 102 may be configured to be associated with and/or operated by an automated devices for example including but not limited to a robotic arms or the like advancing device, for example shown in the form of auxiliary device 55. For example, body 102 may be configured to associate with a robotic arm or the like motorized advancing tool so as to facilitate advancing at least a portion of device 100 toward an insemination site and/or a sample delivery site.

In embodiments device 100 comprises a fluid flow module 104 configured to both receive and/or deliver a bodily fluid sample, most preferably in the form of a semen sample. In embodiments fluid flow module 104 may be configured to receive a semen sample by way of suctioning and/or up-taking a semen sample from a receptacle, in preparation for delivery. In embodiments fluid flow module 104 may be configured to deliver a semen sample to a delivery site in the uterus and/or the intrauterine space by way of mobilizing and/or ejecting and/or discharging a semen sample from a receptacle of device to the delivery site.

In embodiments fluid flow module 104 may therefore comprise at least one or both of a negative pressure sub-module 104a, configured for generating suctioning and/or vacuum to facilitate sample uptake, and/or a positive pressure sub-module 104b configured for ejecting and/or delivering the semen sample to the intrauterine delivery site.

In embodiments fluid flow module 104, 104a, 104b may comprise but is not limited to at least one or more of: a motors, hydraulic motor, electromagnetic motor, piston, air piston, spring loaded movement, gear, linear spring, windings, electromagnet, pump, compressor, the like or any combination thereof.

In embodiments IUI device 100 comprises a sample channel 106 that is configured to receive and/or associate with a semen sample configured to be delivered with device 100. In embodiments sample channel 106 may be configured as an elongated tube that may directly uptake and/or receive a semen sample for delivery.

In embodiments sample channel 106 may be configured in the form of a sheath and/or guiding sheath for receiving a catheter or the like receptacle already loaded with a semen sample for delivery.

In embodiments at least a portion of channel 106 may feature at least one or more sensors of sensor module 120.

In embodiments IUI device comprises electronic module 110 comprising electronics circuitry, hardware, software to render device 100 functional and operational.

In embodiments electronics module 110 may comprise a plurality of optional sub-modules for example including but not limited to a power supply module 112, controller and/or processor module 114, user interface module 111, and memory module 118. In a preferred embodiments electronics module 110 may further comprise a communication module 116.

In embodiments electronics module 110 may be functionally associated with and/or comprise a sensor module 120.

In embodiments, User Interface (UI) module 111 may provide a user with means for interfacing with device 100 preferably via processor module 114. User interface 111 may be provided in the form of an activation button, audiovisual display, the like or any combination thereof. In embodiments, UI module 111 may be provided in optional forms for example including but not limited to buttons, dials, displays, alphanumeric display, touch screen, touch pad, buzzer, tactile pad, at least one light emitting diode (LED), at least one organic LED (OLED), speakers, microphone, or any combination thereof.

In embodiments processor module 114 provides the necessary processing hardware and/or software necessary to render device 100 functional. In embodiments controller and/or processor module 114 may provide for controlling any portion of device 100 and in particular fluid flow module 104 and sensor module 120. For example, processor 114 may be utilized to determine the status of a semen sample associated with channel 106 with at least one or more sensor of sensor module 120 so as to determine how to control fluid flow module 104. For example, a non-limiting example, a sensed event may for example include but is not limited to sensing the motility and/or pH of a semen sample with sensor module 120 so as to determine the activation level of fluid flow module 104 during delivery.

In embodiments power module 112 provides the necessary hardware and/or software to power device 100 therein rendering device 100 operational. Power module 112 may for example be provided in optional forms for example including but not limited to battery, rechargeable induction battery, induction coil, capacitors, super capacitors, inductors the like power source or any combination thereof.

In embodiments communication module 116 preferably provides the necessary hardware and/or software to facilitate communication for device 100 and in particular with data processing center 50 forming system 150. Optionally, communication module 116 may be utilized to communicate with optional auxiliary devices 55 (shown in a broken line). For example, an auxiliary device may for example include but is not limited to a smartphone, mobile processing and communication device, imaging device, server, computer, healthcare service provider dedicated system, first respondent call center, health care call center, the like or any combination thereof.

In some embodiments communications module 116 may be utilize various communication protocols for example including but not limited to wireless communication, cellular communication, wired communication, near field communication, BLUETOOTH, ZIGBEE, optical communication, the like and/or any combination thereof.

In embodiments memory module 118 provides the necessary hardware and/or software to facilitate operations of device 100 by enabling storing and/or retrieving stored data and/or the like as is known in the art.

In embodiments sensor module 120 provides the necessary hardware and/or software to facilitate operations of at least one or more sensor(s) associated with device 100 to enable sensing various events in and around device 100 both internal and/o external to device 100. For example, in some embodiments sensor module 120 may be configured to sense the internal environment by sensing the environment internal to sampling channel 106. In some embodiments, for example, sensor module 120 may be configured to sense the environment external to device 100, for example, the intrauterine space and in particular the delivery site in the intrauterine space.

In embodiments sensor module 120 may comprise at least one or more sensor selected from the group consisting of: image sensor 126, temperature sensor 122, pH sensor 124, flow-meter 128, pulse oximeter, pressure sensor, acoustic sensor, microphone, positional sensor 125, the like or any combination thereof.

In embodiments sensor module 120 may be disposed along any portion of device 100, and in particular channel 106 and/or body 102.

In embodiments image sensor 126 may be provided in optional forms for example including but not limited to camera that provides imagery of a distal end of sample channel 106. In some embodiments image sensor may be provided in the form of ultrasound sensor, that may optionally be disposed along a portion of body 102.

In some embodiments at least a portion of channel 106 may feature an image sensor 126 in the form of a camera, for example for facilitating and/or determining placement and/or location of a sample within the intrauterine space by way of visual assistance with an image sensor 126 in the form of a camera.

In embodiments an image sensor 126, optionally in the form of a camera, may be configured to provide image processing and/or analysis so as to infer and/or obtain parametric data relating to the sample associated with device 100 via channel 106. For example, such image processing analysis may provide to determine at least one parameter for example including but not limited to: sample volume, concentration of semen within sample, semen morphology, semen motility within sample, the like parameters associated with the sample or its contents, any combination thereof.

In some embodiments sensor module 120 may comprise at least one position sensor 125 configured to identify the position a sample channel 106 so as to convey the spatial location of a semen sample within the intrauterine space. In embodiments position sensor 125 may be provided in optional forms for example including but not limited to optical sensors, mechanical sensors, electromagnetic sensor, induction sensor, magnetic based sensors or the like sensor provided for determining and the relative position of sample and/or sample channel 106 within the uterus.

In embodiments system 150 comprises IUI device 100 that is in communication with a data processing center 50. In embodiments data processing center 50 may be provided in optional forms for example including but not limited to a server, computer, smartphone, or the like processing and communication device capable of receiving and/or exchanging data from device 100 so as to process the data. In embodiments, processing center 50 is preferably utilized to apply big data algorithms and/or the like artificial intelligence algorithms to analyze data received from IUI device 100. Optionally communication between processing center 50 and IUI device 100 is facilitated with communication module 116 utilizing any communication protocol for example including wireless, cellular, wired, near field, the like or any combination thereof.

In embodiments system 150 and/or device 100 and/or data center 50 may communicate with additional auxiliary devices 55 to undertake processing of data associated with system 150 and/or device 100. For example, such auxiliary device in the form of communication devices may for example include but is not limited to a smartphone, mobile processing and communication device, imaging device, server, computer, healthcare service provider dedicated system, first respondent call center, health care call center, the like or any combination thereof.

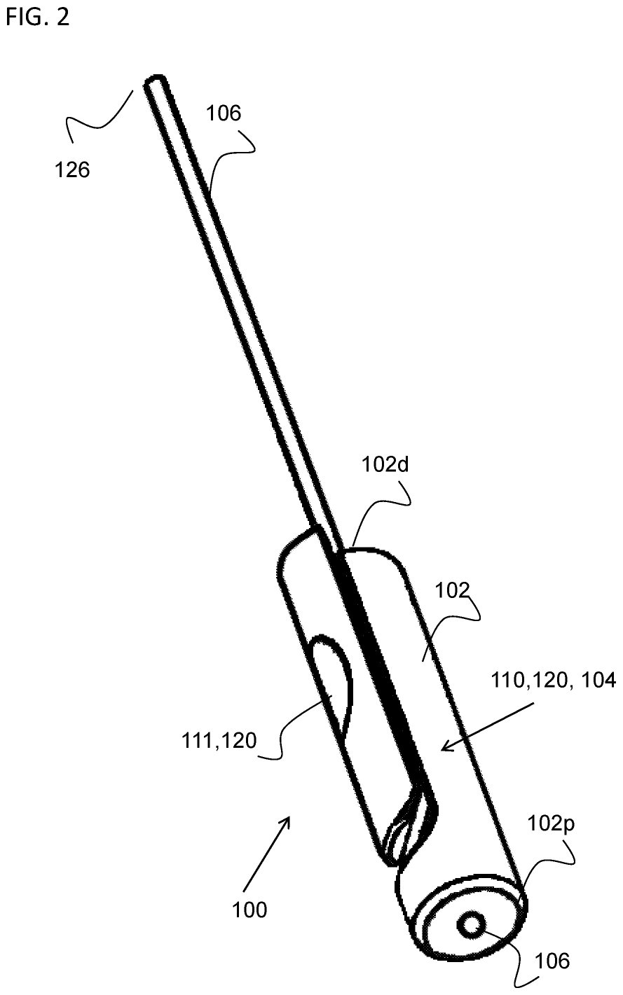

FIG. 2 shows a schematic illustrative diagram of a non-limiting example of a configuration of device 100 having a body 102 in the form of a handle. Body 102 has an internal volume for housing electronic module 110, and fluid flow module 104. Body 102 further features a central channel 106 for receiving a sperm sample, that may be analyzed via at least one or more sensors of sensor module 120, and/or further analyzed via data center 50 and/or auxiliary device 55 (see FIG. 1).

Body 102 features a user interface 111 in the form of an activation button to facilitate operation of device 100 both when up-taking and/or receiving a sample into channel 106, with fluid flow module 104, for example via a negative pressure module 104a, and when delivering a sample to an intrauterine location via module 104 more specifically positive pressure 104b.

In embodiments at least one or more sensors of sensor module 120 may be disposed along body 102. For example, an image sensor 126 in the form of a camera may be associated with the distal end of channel 106, for example a shown, so as to facilitate navigating device 100 and in particular channel 106 within the body.

In some embodiments body 102 may be configured to have a proximal end 102p and a distal end 102d, for example as shown, defining a handle for manual manipulation. In some embodiment channel 106 may be configured as a sheath that is defined between body distal end 102d and proximal end 102p.

In some embodiment proximal end 102p may be configured to receive an external insemination tube already containing a sample.

In some embodiments distal end 102d may be configured and/or shaped to accommodate and/or compliment additional tools and/or devices to facilitate an insemination procedure. For example, a distal end 102d may be configured to associate with at least one or more gynecological tools, for example including but not limited to a speculum or the like, to facilitate placement of a distal end of channel 106 within the uterus.

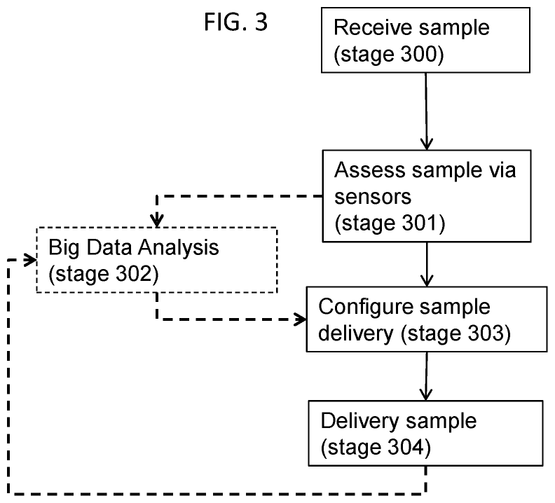

FIG. 3 shows a flowchart of a method for facilitating intrauterine insemination with IUI device 100 according to embodiments of the present invention. In embodiments, in stage 300, device 100 receives a semen sample with channel 106, preferably with the assistance of fluid flow module 104.

Next in stage 301, the semen sample undergoes assessment utilizing at least one or more sensors of sensor module 120. For example, the samples semen motility maybe determined with image sensor 126, the sample's pH may be determined with pH sensor 124, the sample's temperature may be determined with temperature sensor 122, the sample viscosity and/or general flow characteristics may be ascertained with flow sensor 128, or the like.

Next in optional stage 302, the data analyzed by device 100 may be communicated to a higher processing center, for example including but not limited to data center 50 and/or other auxiliary (55) processing centers or devices, for undertaking big data analysis. Optionally, the analysis may be performed offline (irrespective to the timing of the IUI procedure) and/or in real-time during the IUI procedure. If preformed in real time data analysis performed by data center 50 may be communicated back to a user of IUI device 100, for example via communication module 116 and displayed to a user via display user interface 111. Optionally, data center 50 may communication to device 100 a go/no-go signal based on the quality of the sample. For example, if data center 50 analysis determines that the sample is not of sufficient quality or low likelihood of success, a “stop signal” may be communicated to IUI device 100 so as to terminate the procedure.

Next in stage 303, the data assessment from stage 301 and the optional data analysis from stage 302, may be utilized to configure the delivery parameters of the sample. Preferably sample parameters identified with device 100 may be utilized to configure the location, timing, delivery rate, flow rate of the sample into the delivery site. For example, the samples semen motility and/or flow parameters may be utilized to determine how deep to deliver the sample within the uterus. For example, the samples semen motility and/or flow parameters may be utilized to configure to adjust and/or control the fluid flow module 104 and in particular positive pressure sub-module 104b to determine the delivery rate and sample ejection speed and/or force, or the like parameters.

Next in stage 304 the sample is delivered to the intrauterine space. Optionally additionally offline parameters after the delivery process, for example success rate, may be communicated to data center 50 and/or an auxiliary (55) processing center for further analysis so as to facilitate future IUI procedures for analysis and/or decision making.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely for the purpose of improving the intelligibility of the claims and are in no way limiting on the scope of the claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

As used herein the term “about” refers to +/−10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

There are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

While the invention has been described with respect to a limited number of embodiments, it will be appreciated that many variations, modifications and other applications of the invention may be made.

1. An intrauterine insemination (IUI) device (100), the device comprising, a body (102), a sampling channel (106), a sample fluid flow module (104), an electronics module (110), and a sensor module (120), wherein the sensor module (120) is configured to perform an analysis of a sample associated with said sampling channel (106) so as to determine sample delivery parameters, and wherein said channel is in the form selected from at least one of: a catheter and/or sheath and/or guiding sheath; and wherein said sensor module comprises an image sensor (126) that is configured to determine sample parameters associated with a sample disposed in said channel (106).

2. (canceled)

3. The device of claim 2 wherein said channel (106) is configured to feature said mage sensor (126).

4. The device of claim 1 wherein said fluid flow module (104) comprises at least one of: a negative pressure module (104a) configured for receiving a sample into device (100); or a positive pressure module (104b) configured for delivering a sample from device (100) into a delivery site within the intrauterine space.

5. (canceled)

6. (canceled)

7. The device of claim 1 wherein said fluid flow module (104) is provided in the form selected from at least one or more of: motor, hydraulic motor, electromagnetic motor, piston, air piston, spring loaded movement, gear, linear spring, windings, electromagnet, pump, compressor, or any combination thereof.

8. (canceled)

9. (canceled)

10. The device of claim 1 wherein said channel is configured to be a multi-use channel for directly receiving and delivery of a semen sample.

11. The device of claim 1 wherein said channel (106) is configured to receive a disposable and/or single use catheter and/or sleeve and/or sheath.

12. The device of claim 1 wherein said sensor module comprises at least one or more sensor selected from the group comprising: image sensor, pH sensor, flow sensor, temperature sensor, pressure sensor, positional sensor, or any combination thereof.

13. The device of claim 12 wherein said positional sensor (125) is configured to identify the position of a sample channel (106) within the intrauterine space.

14. The device of claim 13 wherein said position sensor (125) is provided in the form selected from at least one or more of: optical sensors, mechanical sensors, electromagnetic sensor, induction sensor, magnetic based sensors.

15. (canceled)

16. The device of claim 1 wherein said body is configured in the form of a handle that is fit with said electronics module, sampling channel and a fluid flow module.

17. The device of claim 1 that is associated with an auxiliary device (55) in the form of a motorized advancing tool or a motorized robotic arm.

18. (canceled)

19. An IUI system (150) comprising the device of claim 1 and in communication with a data processing center (50).

20. The system of claim 19 wherein said data processing center is configured to perform big data analysis wherein such analysis may be utilized to improve treatment success rates, and/or to change treatment protocols, to undertake artificial intelligence (AI) analysis, and/or to review all historic data and/or to provide treatment advice substantially in real time.

21. A method for facilitating intrauterine insemination with a device according to claim 1, the method comprising:
a) utilize a fluid flow module (104) to introduce a fluid sample into a sample channel (106); and
b) assess the fluid sample with at least one or more sensors of a sensor module (120); and wherein at least one of an image sensor (126) or a flow sensor (128/) of said sensor module (120) is utilized to assess the fluid sample internal to the sample channel (106); and wherein said fluid sample assessment is selected from: semen sample motility, semen sample activity rate, sample viscosity, sample volume, concentration of semen within sample, sample pH, semen morphology, or any combination thereof.

22. The method of claim 21 wherein the fluid sample assessment is communicated to a higher processing center (50) and/or auxiliary device (55) for further processing.

23. The method of claim 22 wherein said further processing comprises big data analysis.

24. The method of claim 23 wherein a result of said further processing is communicated from said higher processing center (50) and/or auxiliary device (55) is communicated back to device 100 substantially in real time.

25. The method of claim 21 wherein the assessment is provided in the form of a go/no-go signal.

26. The method of claim 21 wherein the assessment is utilized to configure the delivery parameters of the sample.

27. The method of claim 26 wherein said delivery parameters is utilized to determine at least one or more delivery parameters selected from:
a) delivery site within the intrauterine space;
b) rate of sample delivery; or
c) timing of sample delivery; or
d) sample delivery ejection speed; or
e) sample delivery ejection force;
f) delivery location;
g) delivery timing;
h) delivery rate;
i) flow rate of the sample into the delivery site, or
j) any combination thereof.

28. (canceled)

29. (canceled)

30. (canceled)

31. (canceled)
